(19) United States
(12) Patent Application Publication
(10) Pub. No.: US 2018/0272029 A1
(43) Pub. Date: Sep. 27, 2018

(54) Thermally sprayed ceramic layers

(71) Applicant: CeramTec GmbH, Plochingen (DE)

(72) Inventors: Heinrich WECKER, Eckental (DE); Andreas REMPP, Ostfildern (DE)

(21) Appl. No.: 15/763,557

(22) PCT Filed: Sep. 27, 2016
(86) PCT No.: PCT/EP2016/072978

§ 371 Date: Mar. 27, 2018

(30) Foreign Application Priority Data
Sep. 29, 2015 (DE) 10 2015 218 798.5
Jan. 20, 2016 (DE) 10 2016 200 746.7

(51) Int. Cl. A61L 27/10; A61L 27/04; A61L 27/30; A61L 27/32; A61L 27/56; A61F 2/28; C23C 4/11

(52) U.S. Cl. CPC A61L 27/10; A61L 27/04; A61L 27/306; A61L 27/32; A61L 27/56; A61F 2002/30971; C23C 4/11; A61L 2420/02; A61L 2430/38; A61L 2430/24; A61F 2/28

(57) ABSTRACT

The invention relates to thermally sprayed ceramic layers and a method for the production thereof.

The invention relates to thermally sprayed ceramic layers and a method for the production thereof. The ceramic layers are preferably used for functionalizing medical products, in particular prostheses and implants.

Coatings on medical products, in particular on prostheses and implants, are sufficiently known. Such coatings on prostheses and implants typically consist of metals, for example, titanium, and ceramics such as titanium oxide, tricalcium phosphate, or materials from the group of bioglasses. The medical components in this case are either cemented and comprise an additional bone cement, which is to ensure the bonding to the bone structures, or are cement-free, so that the bonding results due to bone growth (osseoconductivity). The use of metals is generally not unproblematic, however, but rather can have the result that the coating does not continue to adhere to the component surface, but rather delamination of the layer occurs. Moreover, a higher risk of allergies and infection accompanies the use of metallic coatings. In addition, the problem results again and again that with the known layers, graded structures (for example, progression from porous to dense) can only be achieved with difficulty on medical components, or layer structures, which are suitable for certain areas of application, cannot be created without greater effort.

The object of the present invention is therefore to provide a coating which does not have the above-mentioned problems, wherein in particular an improvement of the layer adhesion to prevent delamination and improved biocompatibility are to be achieved. Moreover, the coating is to be suitable both for cemented and also cement-free components and is to result in improved bone growth (osseoconductivity) in the case of cement-free components or in improved chemical bonding of the employed cement to the ceramic layer in the case of cemented components.

The present invention is moreover to enable the achievement of specific desired layer structures and even graded layer structures, whereby custom-tailored layer characteristics can be achieved for different areas of application and the production of patient-specific prostheses or implants would also be possible.

The fundamental object of the present invention is achieved as claimed in claim 1 by the provision of a ceramic layer on a component, wherein the ceramic layer is applied to the surface of the component by means of a thermal spraying method. The spraying of the coating is used in this case in particular for roughening and targeted structuring of the component surface. Preferred embodiments are specified in the dependent claims.

The coating is performed on a component which is preferably a medical product. The component is particularly preferably a prosthesis or an implant. With respect to the prostheses, endoprostheses are preferred, wherein these can be cemented or cement-free. The implant can be in particular a joint surface implant, which is used for the partial replacement of joint surfaces in the human body. The joint surfaces can be, for example, surfaces of the shoulder, hip, knee, elbow, and the entire hand (in particular finger) and foot region.

The component according to the invention can also be used for total disk replacements in the region of the spinal column. A use of the component according to the invention in the region of the fusion (stiffening) in trauma therapy or for the spinal column is also possible.

The component can be both a metallic component or a ceramic component in this case. Therefore, coatings of the same type, i.e., the ceramic material of the component and the coating are of the same type, are also possible. The advantage is that lower probabilities of failure due to layer delamination can be achieved because of identical thermophysical properties.

The coating is created by means of methods of thermal spraying (for example, plasma-based methods in atmosphere or in vacuum), wherein the deposition in vacuum takes place partially from the gas phase, in particular for graded and/or columnar structures, and pressures of <50 mbar prevail, in particular in the coating of cement-free implants.

The layer materials are ceramic materials, preferably the materials of the ceramic layer are pure oxide or mixed oxide ceramics, particularly preferably selected from the group consisting of Al₂O₃, ZrO₂, SiO₂, bioglasses, calcium phosphates (for example, TCP and hydroxylapatite), mixtures of the mentioned materials, the starting materials thereof, and mixtures of the mentioned materials which contain additional additives such as known stabilizing agents from the material group of the rare earth elements, for example, ZrO₂.

The layer structure of the sprayed-on ceramic layer can be both homogeneously dense or graded (progression from one into the other layer structure), i.e., can comprise a progression from porous up to dense structures, for example. The porous structure comprises in particular structures having high open porosity, for example, due to a columnar layer structure having corresponding typical morphology and topography. The porous structures are particularly preferably desirable in cement-free prostheses and implants.

The ceramic layer additionally has a specific roughness of the surface, in particular in cemented prostheses and implants. A roughness Rₐ of >>0.03 μm is particularly preferred. An increased surface area improves the interlocking in the bone and/or in the bone cement and thus ensures improvement of the bonding.

1. A ceramic layer on a component, wherein the ceramic layer is applied to the surface of the component by means of a thermal spraying method.

2. The ceramic layer as claimed in claim 1, wherein the component is a medical product, particularly preferably a prosthesis or an implant.

3. The ceramic layer as claimed in claim 2, wherein the component is a cemented or cement-free endoprosthesis.

4. The ceramic layer as claimed in claim 1, wherein the component is a metallic component or a ceramic component.

5. The ceramic layer as claimed in claim 1, wherein the ceramic layer is created by means of vacuum plasma spraying, preferably at pressures <50 mbar.

6. The ceramic layer as claimed in claim 1, wherein the layer materials are ceramic materials, preferably pure oxide or mixed oxide ceramics, particularly preferably ceramic selected from the group consisting of Al₂O₃, ZrO₂, SiO₂, bioglasses, calcium phosphates (for example, TCP and hydroxylapatite) and mixtures of the mentioned materials and the starting materials thereof.

7. The ceramic layer as claimed in claim 1, wherein the layer structure of the ceramic layer is homogeneously dense, porous, or graded (progression from one into the other layer structure).

8. The ceramic layer as claimed in claim 7, wherein the porous structure comprises a columnar layer structure.

9. The ceramic layer as claimed in claim 7, wherein the dense structure has a specific roughness of the surface, preferably a roughness Ra of >>0.03 μm.

10. A method for producing the ceramic layer as claimed in claim 1, wherein the ceramic layer is created by means of methods of thermal spraying (for example, plasma-based methods in atmosphere or in vacuum).
